(19) United States
(12) Patent Application Publication
(10) Pub. No.: US 2026/0157884 A1
(43) Pub. Date: Jun. 11, 2026

(54) SLEEVE, USE OF AN ULTRASHORT PULSE LAYER, METHOD FOR PROCESSING A SLEEVE BLANK AND METHOD FOR MANUFACTURING A SLEEVE

(71) Applicant: Silcoplast AG, WOLFHALDEN (CH)

(72) Inventors: Philipp Gaus, Steinach (CH); Srecko Marinkovic, Egnach (CH)

(21) Appl. No.: 19/337,043

(22) Filed: Sep. 23, 2025

(30) Foreign Application Priority Data
Sep. 23, 2024 (DE) 10 2024 127 348.8

(51) Int. Cl. A61F 9/007; A61F 9/008

(52) U.S. Cl. CPC A61F 9/00736; A61F 9/008; A61F 2009/00887

(57) ABSTRACT

A sleeve, in particular a silicone sleeve, for sucking off fragments in the operative treatment of cataracts, wherein the sleeve comprises a operative area and a connection area which adjoins the operative area, wherein the operative area is laser-processed at least in sections with a laser beam of an ultra-short pulse laser, in particular a femtosecond laser, so that material has been removed from the operative area by the laser processing, the laser beam having a spot size of 2 to 25 micrometers, preferably 5 to 22 micrometers, particularly preferably 8 to 20 micrometers, and a pulse power of at least 25 watts, preferably at least 30 watts, particularly preferably at least 35 watts, during the laser processing.

### BACKGROUND

The invention relates to a sleeve for sucking off fragments in the operative treatment of cataracts, to the use of an ultra-short pulse laser for creating at least one recess in a sleeve and/or for cutting the sleeve to length, to a method for processing a sleeve blank and to a method for manufacturing a sleeve.

During cataract surgery on the eye, the old lens is destroyed. The resulting fragments are sucked off with the help of specially adapted sleeves. To counteract the vacuum created by the suction, there is typically at least one additional opening, particularly on the side of the sleeve, through which fluid is pushed in. The sleeves must meet the highest requirements. On the one hand, very precise production is required, both in terms of the general geometry of the sleeves and the geometry of the holes in the sleeve. The tube diameter of the sleeve determines the fluid flow through the sleeve. The exact geometry of the opening is also very important. On the other hand, the sleeves must have a high degree of purity, which must be taken into account during the manufacturing process.

Once the sleeves are finished, they are typically provided with corresponding openings and the sleeves are typically cut to a finished size. Both the openings and the cutting to length can be made possible by punching. However, the disadvantage of this is that a specific punching insert and die must be manufactured for each required geometry. Furthermore, it has been shown that the abrasive properties of the silicone material used cause very high wear on the punching edges. It would therefore be desirable to find an alternative way of creating the holes and/or cutting them to length.

One approach would be to create the openings during the injection molding process of the sleeve. However, this has shown that undesirable burrs are created, which then have to be removed in an additional process step. This can result in contamination of the sleeves. Furthermore, cutting the sleeves to length cannot be optimally reproduced in the injection molding process. Another approach would be to use a laser beam from a conventional laser to create the holes and cut the sleeves to length. However, it has been shown that the resulting laser ablation appears to be unacceptable from a medical point of view. The burn-off can leave residue and/or visible discoloration on the sleeve. This makes additional washing of the sleeves necessary, which poses a risk of contamination and has not yet been able to completely remove the burn-off.

It is therefore the object of the present invention to provide a possibility with which corresponding sleeves can be produced without the use of punching tools, or at least with a reduced use of punching tools, and whereby the medical requirements can be met. It would also be desirable to have a way of achieving improved precision during processing, in particular when creating openings and cutting to length.

### SUMMARY OF THE INVENTION

According to the invention, a sleeve, in particular a silicone sleeve, is provided for sucking off fragments during the operative treatment of cataracts. The sleeve comprises an operative area and a connection area which adjoins the operative area, wherein the operative area is laser-processed at least in sections with a laser beam of an ultra-short pulse laser, in particular a femtosecond laser, so that material has been removed from the operative area by the laser processing, wherein the laser beam has or has had a spot size of 2 to 25 micrometers, preferably 5 to 22 micrometers, particularly preferably 8 to 20 micrometers, and a pulse power of at least 25 watts, preferably at least 30 watts, particularly preferably at least 35 watts, during laser processing has exhibited. In the context of the present invention, a sleeve is generally understood to mean a sleeve which is intended or suitable for sucking off fragments in the operative treatment of cataracts. It may be particularly advantageous if the sleeve is made of a silicone material. Optionally, the sleeve can be made of a transparent silicone material. A silicone material is in particular a material that consists at least partially of silicone or comprises silicone. Alternatively, the sleeve can also be made of another material. For example, sleeves were previously made from titanium. Silicone has the advantage with respect to titanium that the risk of injury can be reduced due to its lower hardness. The silicone material can be made of molded silicone and/or liquid silicone, for example. The sleeve can comprise at least one lateral opening, in particular a cross hole. Preferably, the at least one lateral opening has rounded corners and/or is oval, in particular round, in shape. The sleeve has an operative area and a connection area. In particular, the at least one lateral opening can be provided at the side of the operative area. The operative area adjoins the connection area. In particular, the operative area can merge into the connection area. Preferably, the operative area has a smaller diameter than the connection area. For example, the operative area can have an outer cross-sectional diameter of 0.5 mm to 4 mm, preferably 1 mm to 2 mm. A ratio of an inner cross-sectional diameter of the operative area to the outer cross-sectional diameter of the operative area can be, for example, 0.3 to 0.95, preferably 0.5 to 0.9, particularly preferably 0.70 to 0.87. For example, the connection area can have an outer cross-sectional diameter of 5 mm to 15 mm, preferably 6 mm to 10 mm. A ratio of an inner cross-sectional diameter of the connection area to the outer cross-sectional diameter of the connection area can be, for example, 0.2 to 0.8, preferably 0.4 to 0.75, particularly preferably 0.5 to 0.65. It may be provided that a ratio of the length of the operative area to the length of the connection area is between 0.5 to 2, preferably 0.8 to 1.2. Corresponding dimensions can be particularly advantageous for the purpose of sucking fragments off. In particular, a particularly good insertion of the sleeve with good suction properties can be achieved. The connection area can be configured so that a line, for example a hose, can be connected to the connection area. In particular, it can be provided that the line, especially the hose, can be slipped over the connection area and/or can be inserted into the connection area, for example plugged or screwed in. The connection area can have a ribbed area on the inner or outer side for attaching a line, in particular a hose. The connection area can have an internal thread, for example for screwing in a connection hose and/or cable. The operative area has been laser-processed at least in sections with a laser beam with a spot size or laser spot size of 2 to 25 micrometers, preferably 5 to 22 micrometers, particularly preferably 8 to 20 micrometers of an ultra-short pulse laser, in particular a femtosecond laser, with a pulse power of at least 25 watts, preferably at least 30 watts, particularly preferably at least 35 watts. Ultra-short pulse lasers are, in particular, laser beam sources that emit pulsed laser light with pulse durations in the range of fractions of a second. Accordingly, a femtosecond laser emits laser light with pulse durations in the femtosecond range. In the context of the invention, laser processing was used to remove material from the operative area of the sleeve. For example, a recess caused by laser drilling or, preferably, laser cutting can be provided. In the case of laser drilling, so much energy is applied locally to the area to be processed that the material at this point is melted and partially vaporized. A contour is cut out during laser cutting. In particular, the contour can correspond to an edge of the recess. In particular, the recess can be created by the laser beam tracing the contour until the material is completely cut through along the contour. Laser cutting can be pulsed or in a continuous wave mode. (continuous wave). In a continuous wave mode, the laser beam is applied continuously and with an essentially constant amplitude. Laser cutting can in particular be remote laser cutting. Advantageously, the parameters according to the invention, in particular the pulse power and the spot size, can at least largely prevent melting of the material at the edge of the recess. Additionally or alternatively, the sleeve can be cut to length by the energy of the laser beam. While previous experiments regularly encountered the problem that excessive burn-off occurred during laser processing of silicone sleeves, so that laser processing of sleeves intended for the purpose according to the invention was considered disadvantageous and rather impractical, it has surprisingly been shown that burn-off can be prevented to such an extent that the required hygienic conditions can be achieved with the laser configuration given in accordance with the requirements. Due to the small spot size on the one hand and high energy on the other, the energy of the laser beam can be bundled and carried out with a sufficiently short pulse duration so that the surroundings of the processed section of the sleeve are only insignificantly affected. In particular, this also results in only a small heat-affected zone and little melting or lateral smouldering, so that hardly any resolidified material is produced around the processed area. Microcracks can also be better prevented and the surface quality of the sleeve can be significantly improved, especially in the area processed by the laser beam. By processing with a laser beam according to the invention, the manufactured sleeve can be characterized in particular by a lack of or only minimal burn-off with high precision of the processed area. The lack of or very slight burn-off can be recognizable in the finished sleeve by the absence of residues, for example chemical residues, absence of traces of smoke, absence of visible discoloration. In particular, there are also no undesirable burrs. A sleeve according to the invention can be produced particularly cost-effectively, especially compared to punching holes or cutting to length by punching. Different versions of sleeves can also be produced with relatively little effort, in particular with different recesses, with regard to the geometry of the recesses.

Advantageously, the operative area comprises a hole or a bore, in particular a lateral hole or lateral bore, which has been produced during laser processing by laser cutting with the laser beam. Alternatively, or additionally, an end of the operative area has been cut to length with the laser beam during laser processing. Advantageously, a geometric design of the bore or hole can be particularly easy to adapt, for example according to use-specific requirements. It has been shown that with the type of laser according to the invention, i.e. in particular with the spot size and pulse power according to the invention, a lateral bore/hole and/or a cut to length can be provided particularly efficiently, precisely and at least largely residue-free. With the laser processing according to the invention, a diameter of the bore or hole can be adapted particularly precisely. The bore or hole can preferably have an oval, optionally round, cross-section and/or rounded corners.

Advantageously, a laser beam with a wavelength of 200 to 500 nm, preferably 300 to 400 nm, most preferably 330 to 380 nm, has been used. A wavelength in the range of 300 to 400 nm has proven to be particularly favorable for being able to process sleeves of commonly used silicone materials particularly efficiently. A wavelength of 330 to 380 nm can enable particularly precise results. With a wavelength in these ranges, focusing to a relatively small laser spot is possible. With a small laser spot, the laser energy can be concentrated on a particularly small focal spot, which makes it possible in particular to process fine contours. The aforementioned wavelength range of 330 to 380 nm is particularly favorable because, on the one hand, it enables particularly precise work, while at the same time it is still relatively easy to provide such a wavelength (with respect to even shorter wavelengths).

Advantageously, a module for frequency multiplication is used to adjust the wavelength of the laser beam. By using a frequency multiplication module, a laser with a natively higher wavelength can advantageously be used, which can significantly improve the availability of suitable lasers, while at the same time a suitably short wavelength can act on the sleeve material. A frequency tripling can also be referred to as THG (third harmonic generation) for short.

Advantageously, the ultra-short pulse laser used is configured to generate laser light in the infrared range, in particular with a wavelength of at least 900 nm, preferably between 900 and 1500 nm, most preferably between 1000 and 1200 nm, whereby a module for frequency multiplication, in particular frequency tripling, was used to adjust the wavelength of the laser beam. A wavelength in this range, in particular above 900 nm, can be produced particularly well and also cheaply, because many industrially used readers operate in this wavelength range. This means that a reader with suitable properties, in particular a corresponding spot size and a suitable pulse power, can be provided relatively easily and inexpensively, while at the same time an advantageously low wavelength can be generated by frequency multiplication. A wavelength range between 1000 and 1200 nm is particularly advantageous, especially in combination with frequency tripling, because this combination can be used to generate a wavelength that is particularly favorable for the production of the required sleeve by means of a relatively readily available reader. For example, the ultra-short pulse laser can generate a wavelength in the order of 1060 nm and the frequency multiplication module reduces the wavelength to a wavelength in the range of 355 nm.

Advantageously, the laser beam used during laser processing has a pulse energy of at least 120 microjoules, preferably at least 150 microjoules, particularly preferably at least 180 microjoules. In other words, the operative area can be laser-processed with a laser beam with a pulse energy of at least 120 microjoules, preferably at least 150 microjoules, particularly preferably at least 180 microjoules.

Advantageously, the laser beam used during laser processing has a diffraction index M²<1,2. In particular, the diffraction index can define the beam quality K to K=1/M². A theoretically optimum beam quality is available at K=1 and M²=1. Accordingly, a lower diffraction index M² means a higher beam quality. A lower diffraction index allows better focusing of the laser beam by focusing optics, e.g. by an optical lens, and provides a measure of how close a laser beam is to an ideal Gaussian beam. The diffraction index can be determined in accordance with ISO/DIS 11146, for example. It has been shown that burn-off can be prevented particularly well with a measurement number M²<1,2. In particular, particularly good focusability can be achieved with such a value. The dimensional value can be set or achieved in particular by the geometry of the laser resonator.

Advantageously, the sleeve is manufactured in such a way that a laser smoke suction device is used during the processing of the sleeve with the laser beam in order to suck off any laser ablation that occurs. Preferably, the laser smoke suction device can comprise an suction fan. The suction fan can have a power of 0.5 Kw to 5 Kw, preferably 0.8 Kw to 2 Kw. The suction fan can have an air flow rate of at least 100 m³/h, preferably at least 200 m³/h, particularly preferably 200 to 400 m³/h. It has been found that with this power and this air flow rate, a very efficient sucking of the remaining burn-off can be achieved. Particularly with 0.8 Kw to 2 Kw suction power and an air flow rate of 200 to 400 m³/h, virtually residue-free laser processing can be achieved.

Advantageously, the operative area comprises a first tube-like hollow section with a lateral hole or a lateral bore, wherein the lateral hole or the lateral bore has been produced by laser cutting with the laser beam. In particular, the first tube-like hollow section can be configured so that it can be used to transport aspirated fragments, which are produced in particular during the treatment of cataracts, and/or so that it can be used to add fluid for pressure equalization. For example, a ratio of one of the inner diameters of the first tube-like hollow section to an outer diameter of the first tube-like hollow section can be in the range of 0.5-0.0.99, preferably 0.6 to 0.95, particularly preferably 0.8 to 0.90. This means that particularly good suction properties can be achieved.

Advantageously, the connection area of the sleeve comprises a second tube-like hollow section, wherein the second tube-like hollow section has a larger cross-sectional diameter, in particular an outer cross-sectional diameter, than the first tube-like hollow section, in particular a cross-sectional diameter at least twice as large, preferably at least three times as large. Particularly preferably, an inner cross-sectional diameter of the second tube-like hollow section can be in a ratio of 0.2 to 0.3 to an inner cross-sectional diameter of the first tube-like hollow section. Particularly preferably, an outer cross-sectional diameter of the second tube-like hollow section to an outer cross-sectional diameter of the first tube-like hollow section may be in a ratio of 0.1 to 0.25. Preferably, it may be provided that the axes of the first tube-like hollow section and the second tube-like hollow section are parallel to each other. In particular, the first tube-like hollow section may be arranged substantially concentrically with the second tube-like hollow section. For example, a ratio of an inner diameter of the second tube-like hollow section to an outer diameter of the second tube-like hollow section can be in the range of 0.2-0.8, preferably 0.4 to 0.75, particularly preferably 0.5 to 0.7. This means that, on the one hand, particularly good suction properties can be achieved and, on the other hand, a connection, in particular a connection hose, can be attached particularly well to the connection area.

Advantageously, a ratio of the outer diameter of the first tube-like hollow section to an inner diameter of the first tube-like hollow section is lower than a ratio of the outer diameter of the second tube-like hollow section to an inner diameter of the second tube-like hollow section. In particular, a ratio of the outer diameter of the first tube-like hollow section to an inner diameter of the first tube-like hollow section can be lower by a factor in the range from 0.5 to 0.9, preferably 0.6 to 0.8, than a ratio of the outer diameter of the second tube-like hollow section to an inner diameter of the second tube-like hollow section. This makes it possible to achieve particularly favorable flow properties in the sleeve, especially with regard to the connection of a connection hose to the connection area.

Advantageously, the connection area has a transition area between the first tube-like hollow section and the second tube-like hollow section, so that the first tube-like hollow section merges into the second tube-like hollow section, wherein in particular an inner hollow area of the first tube-like hollow section merges into an inner hollow area of the second tube-like hollow section. Preferably, the transition area can be substantially concentric to the first tube-like hollow section and to the second tube-like hollow section.

Advantageously, the connection area and the operative area are a common one-piece body. This enables particularly simple manufacture, for example by injection molding. Furthermore, good stability and a good density of the entire sleeve can be achieved.

Advantageously, the sleeve is injection molded. An injection molding process can be a particularly simple way to produce a sleeve or a sleeve blank that is still laser machined.

Advantageously, the sleeve is made of a transparent silicone material, in particular injection molded. A sleeve made of transparent silicone material can be particularly advantageous when used as an operative tool for the treatment of cataracts.

A further aspect of the invention is the use of an ultra-short pulse laser, in particular a femtosecond laser, for removing material by laser processing from a sleeve blank, in particular a silicone sleeve blank, in particular creating at least one recess, in particular a hole or bore, in the sleeve blank. The ultra-short pulse laser used generates a laser beam with a spot size of 2 to 25 micrometers, preferably 5 to 22 micrometers, particularly preferably 8 to 20 micrometers, and a pulse power of at least 25 watts, preferably at least 30 watts, particularly preferably at least 35 watts, in the manufacture of a sleeve, particularly a silicone sleeve, for sucking off fragments in the operative treatment of cataracts.

A further aspect of the invention a method for processing a sleeve blank, in particular a silicone sleeve blank, for sucking off fragments in the operative treatment of cataract to remove material from the sleeve blank, in particular to cut the sleeve blank to length and/or provide it with at least one recess, the method comprising:
providing the sleeve blank
focusing a laser beam on at least one predetermined focus point on the sleeve blank, so that material is removed from the sleeve blank at the at least one focus point with the aid of the laser energy, the sleeve blank being cut to length in particular with the aid of the laser energy and/or being provided with a recess, in particular a hole or bore; the laser beam being generated by an ultrashort pulse laser, in particular a femtosecond laser, the laser beam having a spot size of 2 to 25 micrometers. The laser beam is generated by an ultra-short pulse laser, in particular a femtosecond laser, wherein the laser beam has a spot size of 2 to 25 micrometers, preferably 5 to 22 micrometers, particularly preferably 8 to 20 micrometers, and a pulse power of at least 25 watts, preferably at least 30 watts, particularly preferably at least 35 watts. In particular, the method can be used to produce a sleeve according to the invention from the sleeve blank. The term sleeve blank is to be understood broadly in the context of the present invention. In particular, it generally refers to a sleeve in a manufacturing stage before laser processing or before the addition of corresponding holes/recesses and/or before cutting to length. The removal of laser material can preferably be computer-controlled in an automated manner, in particular by controlling the laser beam and the positioning of the laser beam by a computer program. The computer program can include adjustable parameters that can be adapted to user-specific requirements. For example, the computer program can be configured so that the geometry of a recess, in particular a hole, created by laser cutting, in particular the depth, shape and/or cross-section, can be adjusted. The recess, in particular the hole, can be created in particular by laser cutting with the laser beam. During laser cutting, the laser traces a contour of the intended hole until the material is completely cut through. Advantageously, the parameters according to the invention, in particular the pulse power and the spot size, can at least largely prevent the material from melting at the edge of the recess or hole. The cutting to length can be provided in particular by laser beam cutting. Preferably, pulsed operation can be provided for the laser beam, in particular with a pulse frequency in the order of 1 Hz to 1 MHz. The ultra-short pulse laser used is preferably a femtosecond laser. For example, a pulse duration of 50 fs to 800 fs, preferably 100 fs to 500 fs, particularly preferably 200 to 300 fs, can be used. Preferably, a pulse energy of the laser beam is at least 120 microjoules, preferably at least 150 microjoules, particularly preferably at least 180 microjoules. Advantageously, the laser beam used can have a wavelength of 200 to 500 nm, preferably 300 to 400 nm, most preferably 330 to 380 nm. Preferably, the ultra-short pulse laser used is configured such that the laser beam striking the focus point has a diffraction index M²<1,2. Preferably, the ultra-short pulse laser used can be operated with cooling, in particular water cooling. Preferably, a laser smoke suction device is used during the treatment of the sleeve blank with the laser beam in order to suck off any laser ablation that occurs. The sleeve blank used is preferably made of a silicone material. In other words, the sleeve blank can be a silicone sleeve blank. The core blank can optionally be made of a transparent silicone material. Preferably, the sleeve blank can be injection molded. Preferably, laser parameters, in particular comprising a power setting, a frequency and/or a scanning speed, are optimized for the process. The laser parameters can be determined on a trial basis and stored for further use.

Advantageously, a frequency multiplication module is used to adjust the wavelength of the laser beam. In particular, a laser with a higher wavelength can be used than the wavelength of the laser beam that is focused on the core blank or that hits the core blank.

Advantageously, the ultra-short pulse laser is configured to generate laser light in the infrared range, in particular with a wavelength of at least 900 nm, preferably between 900 and 1500 nm, most preferably between 1000 and 1200 nm, wherein a module for frequency multiplication, in particular frequency triplication, is used to adjust the wavelength of the laser beam that strikes the at least one focus point, in particular to a lower wavelength value. Preferably, the wavelength of the laser beam can be set to a wavelength of 200 to 500 nm, preferably 300 to 400 nm, most preferably 330 to 380 nm.

Advantageously, the ultra-short pulse laser comprises a lens system comprising, in particular, high-purity, all-quartz lenses. Full quartz lenses can be particularly suitable for focusing the laser beam onto a surface of the workpiece or sleeve. Low energy losses, in particular due to high transmission and low absorption, can be achieved with solid quartz lenses.

Advantageously, the sleeve blank comprises at least one first tube-like hollow section, wherein the at least one focus point is directed onto a side wall of the first tube-like hollow section, wherein the recess is produced as a lateral bore in the tube-like hollow section by the laser beam.

Advantageously, the sleeve further comprises at least a second tube-like hollow section, wherein the second tube-like hollow section has a larger cross-sectional diameter than the first tube-like hollow section, in particular a cross-sectional diameter at least twice as large, preferably at least three times as large.

A further aspect of the invention is a method of manufacturing a sleeve, in particular a silicone sleeve, for sucking off fragments in the operative treatment of cataracts, comprising the following steps:
Manufacturing a sleeve blank, in particular from a silicone material, in particular injection molding the sleeve blank in an injection molding process,
processing the injection molded sleeve blank using a method for processing a sleeve blank as described herein in order to cut the sleeve to length and/or provide it with at least one recess. A silicone sleeve is to be understood in particular as a sleeve which is made of a silicone material, in particular injection molded. Laser processing of the injection molded sleeve blank can immediately follow injection molding and/or be directly integrated into the injection molding process. Optionally, the sleeve blank can be injection molded from a transparent silicone material.

Advantageously, the sleeve blank is injection molded in such a way that it comprises at least one first tube-like hollow section, wherein the at least one focus point is directed onto a side wall of the first tube-like hollow section, wherein the recess is produced as a lateral bore in the tube-like hollow section by the laser beam.

Advantageously, the sleeve is injection molded in such a way that it further comprises at least a second tube-like hollow section, and that the second tube-like hollow section has a larger cross-sectional diameter than the first tube-like hollow section, in particular a cross-sectional diameter at least twice as large, preferably at least three times as large.

Advantageously, the sleeve is injection molded in such a way that a transition area between the first tube-like hollow section and the second tube-like hollow section is furthermore produced, so that the first tube-like hollow section merges into the second tube-like hollow section, wherein in particular an inner hollow area of the first tube-like hollow section merges into an inner hollow area of the second tube-like hollow section.

Advantageously, the sleeve is injection molded such that a ratio of the diameter of the outer wall of the first tube-like hollow section to an inner diameter of the first tube-like hollow section is less than a ratio of the diameter of the outer wall of the second tube-like hollow section to an inner diameter of the second tube-like hollow section.

The features and advantages described herein in the context of one aspect of the invention can also be transferred to the respective other aspects of the invention. For example, the method for manufacturing a sleeve may also have corresponding features and advantages such as the sleeve, the use and the method for processing a sleeve blank. Conversely, the sleeve according to the invention, the use according to the invention and the method for processing a sleeve blank according to the invention may also have corresponding features and advantages as the method for manufacturing a sleeve or as the other of these aspects.

### BRIEF DESCRIPTION OF THE DRAWINGS

Further advantages and features of the present invention are apparent from the following description with reference to the figures. Individual features disclosed in the embodiments shown may also be used in other embodiments, unless expressly excluded. It shows

FIG. 1 is a sleeve for sucking off fragments in the operative treatment of cataracts according to one embodiment of the invention;

FIG. 2 is an enlarged view of the section of the sleeve of FIG. 1 marked with a circle;

FIG. 3 is the section of FIG. 2 in an alternative embodiment;

FIG. 4 is a sleeve 1 for sucking off fragments in the operative treatment of cataracts according to a further embodiment of the invention;

FIG. 5 is a flowchart of a method for processing a sleeve blank according to one embodiment of the invention; and

FIG. 6 is a flowchart of a method of manufacturing a sleeve for sucking off fragments in the operative treatment of cataracts according to one embodiment of the invention.

### DETAILED DESCRIPTION OF THE PREFERRED EMBODIMENTS

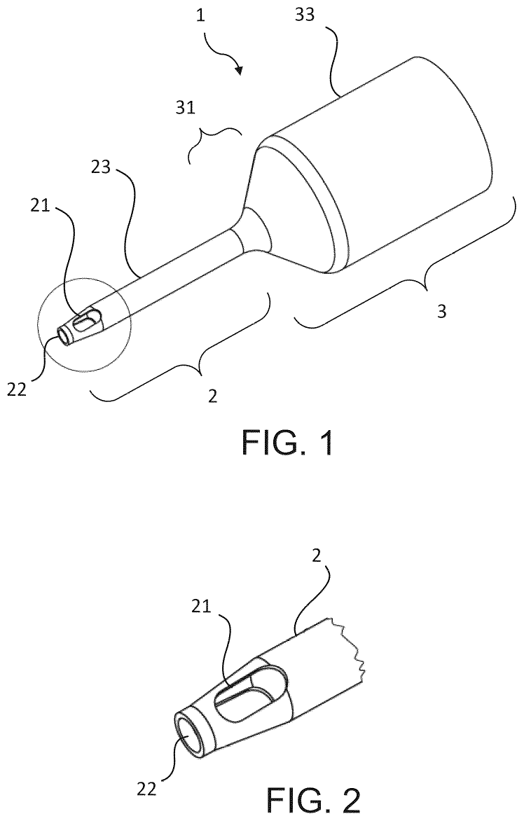

FIG. 1 shows a sleeve 1 for sucking off fragments in the operative treatment of cataracts according to one embodiment of the invention. The sleeve 1 can be, for example, a silicone sleeve, in particular an injection molded silicone sleeve. The sleeve 1 comprises an operative area 2 and a connection area 3 adjoining the operative area 2. Preferably, the connection area 3 and the operative area 2 can be a common one-piece body. The operative area 2 comprises a first tube-like hollow section 23 through which aspirated fragments of the lens can be transported during treatment. The operative area 2 has been processed in the anterior section of the first tube-like hollow section 23 with a laser beam of a femtosecond laser, such that material has been removed from the operative area 2 by laser processing, namely laser cutting, to create a lateral hole 21. In addition, an end 22 of the operative area 2 was also cut to length by a laser beam, in particular by laser cutting. For laser cutting a hole, as well as for cutting to length, a laser beam with a spot size of 2 to 25 micrometers, particularly preferably 8 to 20 micrometers, and with a pulse power of at least 25 watts, particularly preferably at least 35 watts, is used according to the invention. It has been found that with these values, residues caused by burn-off on the sleeve in the vicinity of the laser processing can be significantly reduced or even essentially completely avoided. The connection area of the sleeve comprises a second tube-like hollow section 33, which has a larger cross-sectional diameter than the first tube-like hollow section 23 of the operative area 2. In particular, the second tube-like hollow section 33 in this example has an outer cross-sectional diameter approximately five times as large as the first tube-like hollow section 34. The first tube-like hollow section 23 and the second tube-like hollow section 33 are arranged substantially concentrically to each other. The connection area 3 further comprises a transition area 31 which is between the first tube-like hollow section 23 and the second tube-like hollow section 33, so that the first tube-like hollow section 23 merges into the second tube-like hollow section 33 by means of the transition area 31. In particular, an inner hollow area of the first tube-like hollow section 23 also merges into an inner hollow area of the second tube-like hollow section 33. The transition area 31 is essentially concentric to the first tube-like hollow section 23 and to the second tube-like hollow section 33.

FIG. 2 shows an enlarged view of the section of the sleeve 1 of FIG. 1 marked with a circle. The front section of the first tube-like hollow section 23 in which there is a lateral hole 21 can be seen. In this embodiment, the lateral hole 21 has an elongate extension parallel to the longitudinal extension of the first tube-like hollow section 23. In this embodiment, the lateral hole is a cross hole or a cross bore that extends completely through the first tube-like hollow section 23, so that the first tube-like hollow section has an opening on two opposite lateral sides. The corners of the lateral hole 21 are rounded. A front opening can also be seen at the end 22 of the operative area 2.

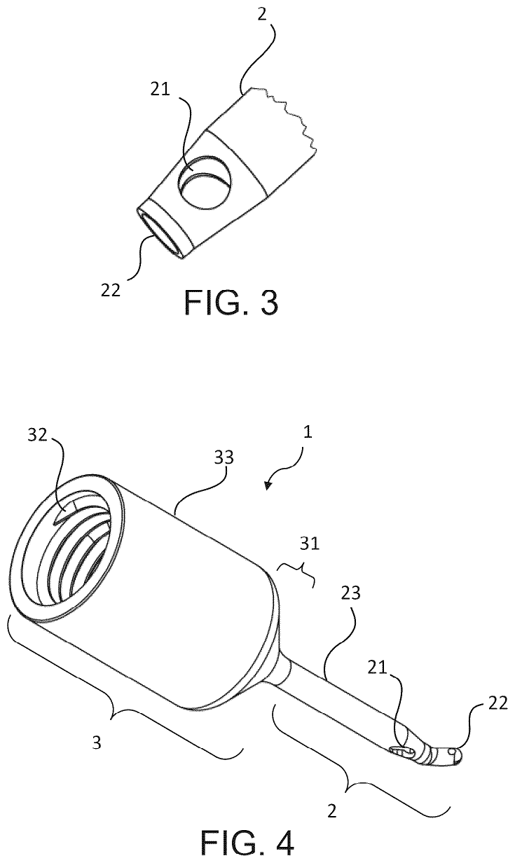

FIG. 3 shows the section of FIG. 2 in an alternative embodiment. In this embodiment, the lateral hole 21 is essentially configured as a round opening. Advantageously, the method according to the invention can be used to adapt the geometric properties of the intended lateral hole 21 with relatively little effort. For example, an adaptation of the hole can be realized by a program adaptation in a program that controls the laser beam.

FIG. 4 shows a sleeve 1 for sucking off fragments during the operative treatment of cataracts according to a further embodiment of the invention. This embodiment differs from that in FIG. 1 by the end 22 of the tube-like hollow section 23. In this embodiment, the end 22 is bent or has an angle. This embodiment can be advantageous for some operational requirements. In this embodiment, an internal thread 32 can also be seen, which is provided in the connection area. Optionally, the embodiment shown in FIG. 1 can also have such or a similar internal thread 32. The internal thread can, for example, be provided for screwing in a connecting hose and/or cable.

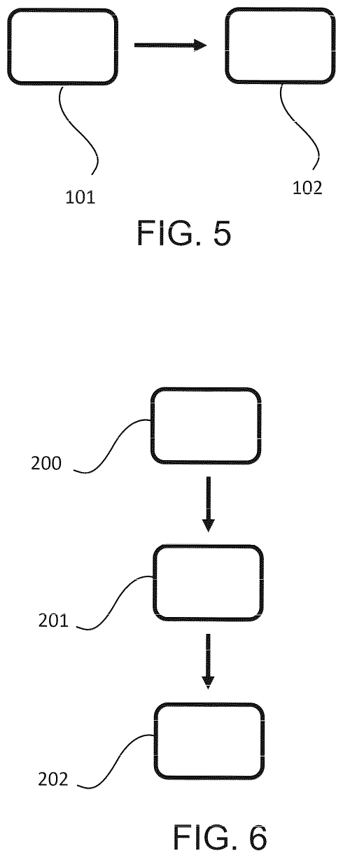

FIG. 5 shows a flow chart of a method for processing a sleeve blank according to an embodiment of the invention. In a first step 101, a sleeve blank is provided. The core blank can preferably be a silicone core blank. In a further step 102, a laser beam is focused on at least one predetermined focus point on the core blank, so that material is removed from the core blank at the at least one focus point with the aid of the laser energy. In particular, the core blank can be cut to length and/or provided with a recess using the laser energy. In particular, the recess can be a lateral hole 21. In this case, the laser beam is generated by an ultra-short pulse laser, in particular a femtosecond laser, wherein the laser beam has a spot size of 2 to 25 micrometers, preferably 5 to 22 micrometers, particularly preferably 8 to 20 micrometers, and a pulse power of at least 25 watts, preferably at least 30 watts, particularly preferably at least 35 watts. Preferably, the laser beam can have a pulse energy of at least 120 microjoules, preferably at least 150 microjoules, particularly preferably at least 180 microjoules. Preferably, the laser beam can have a wavelength of 200 to 500 nm, preferably 300 to 400 nm, most preferably 330 to 380 nm. To adjust the wavelength of the laser beam, a module for frequency multiplication can optionally be used. For example, the ultra-short pulse laser used can be configured to generate laser light in the infrared range, for example preferably between 1000 and 1200 nm. A frequency tripling module can be used to adjust the wavelength of the laser beam that hits the focus point to a desired wavelength value, preferably in the range of 300 nm to 400 nm. Tests at a wavelength of 343 nm have shown that very good results can be achieved with a processing time of 3.3 seconds per hole, whereby a processing time of 1.54 seconds per hole could also be achieved with good results. Preferably, an optional laser smoke suction device can be used during processing of the sleeve with the laser beam in order to suck off any laser ablation that occurs. This can advantageously reduce any remaining laser ablation.

FIG. 6 shows a flowchart of a method for manufacturing a sleeve 1 for sucking off fragments in the operative treatment of cataracts according to one embodiment of the invention. In a first step 200, a sleeve blank is injection molded. In particular, the sleeve blank may be made of a silicone material and the sleeve produced by the method may accordingly be a silicone sleeve. The following steps 201, 202 can optionally be integrated directly into the first step 200 or follow on from it. In a further step 201, the sleeve blank is provided for laser processing. In a still further step 202, a laser beam is focused on at least one predetermined focus point on the core blank, so that material is removed from the core blank at the at least one focus point with the aid of the laser energy. The further steps 201, 202 can essentially correspond to steps 101, 102 of the method described with reference to FIG. 5.

### REFERENCE SIGN LIST

1—sleeve
2—operative area
3—connection area
21—lateral hole
22—end of the operating area
23—first tube-like hollow section
31—transition area
32—internal thread
33—second tube-like hollow section

The invention claimed is:

1. A silicone sleeve for sucking off fragments in the operative treatment of cataracts, comprising:
an operative area; and
a connection area adjoining the operative area;
wherein the operative area is laser-processed at least in sections with a laser beam of an ultra-short pulse laser including a femtosecond laser, so that material has been removed from the operative area by the laser processing; and
wherein the laser beam during laser processing has or has had a spot size of 2 to 25 micrometers and a pulse power of at least 25 watts.

2. The sleeve according to claim 1, wherein the spot size is 5 to 22 micrometers.

3. The sleeve according to claim 2, wherein the spot size is 8 to 20 micrometers.

4. The sleeve according to claim 1, wherein the pulse power is at least 30 watts.

5. The sleeve according to claim 4, wherein the pulse power is at least 35 watts.

6. The sleeve according to claim 1, wherein the operative area comprises a lateral hole or lateral bore, which has been produced during the laser processing by laser cutting with the laser beam and/or wherein an end of the operative area has been cut to length during laser processing with the laser beam.

7. The sleeve according to claim 1, wherein the sleeve is manufactured such that during the processing of the sleeve with the laser beam, a laser smoke suction device has been used to suck off a resulting laser ablation.

8. The sleeve according to claim 1, wherein the operative area comprises a first tube-like hollow section having at least one lateral hole, wherein the lateral hole has been produced by laser cutting with the laser beam.

9. The sleeve according to claim 8, wherein the connection area of the sleeve comprises a second tube-like hollow section having a larger cross-sectional diameter than a cross-sectional diameter of the first tube-like hollow section.

10. The sleeve according to claim 9, wherein the cross-sectional diameter of the second tube-like hollow section is at least twice the cross-sectional diameter of the first tube-like hollow section.

11. The sleeve according to claim 10, wherein the cross-sectional diameter of the second tube-like hollow section is at least three times the cross-sectional diameter of the first tube-like hollow section.

12. The sleeve according to claim 1, wherein the sleeve is injection molded.

13. A method for processing a silicone sleeve blank for sucking off fragments in the operative treatment of cataracts in order to cut the sleeve blank to length and/or to provide the sleeve blank with at least one recess for producing a sleeve according to claim 1, the method comprising:
providing the sleeve blank;
focusing a laser beam on at least one predetermined focus point on the sleeve blank so that material is removed from the sleeve blank at the at least one focus point with the aid of the laser energy, the sleeve blank being cut to length and/or provided with a recess with the aid of the laser energy;
wherein the laser beam is generated by an ultra-short pulse laser including a femtosecond laser; and
wherein the laser beam has a spot size of 2 to 25 micrometers and a pulse power of at least 25 watts.

14. The method of claim 13, wherein the spot size is 5 to 22 micrometers.

15. The method of claim 14, wherein the spot size is 8 to 20 micrometers.

16. The method of claim 13, wherein the pulse power is at least 30 watts.

17. The method of claim 16, wherein the pulse power is at least 35 watts.

18. The method according to claim 13, wherein the ultra-short pulse laser is configured to generate laser light in the infrared range with a wavelength of at least 900 nm, wherein a module for frequency multiplication comprising frequency tripling is used to adjust the wavelength of the laser beam, which strikes the at least one focus point, to a lower wavelength value.

19. The method of claim 18, wherein the wavelength is between 900 and 1500 nm.

20. The method of claim 19, wherein the wavelength is between 1000 and 1200 nm.

21. A method for producing the silicone sleeve, comprising:
injection molding the sleeve blank from a silicone material in an injection molding process; and
processing the injection molded sleeve blank by a method according to claim 13 in order to cut the sleeve to length and/or provide the sleeve with the at least one recess.
